(19) United States
(12) Patent Application Publication
(10) Pub. No.: US 2024/0417708 A1
(43) Pub. Date: Dec. 19, 2024

(54) TAGGED EXOGLYCOSIDASE ENZYMES AND IMMOBILIZED GLYCAN SEQUENCING APPROACH

(71) Applicant: PANNON EGYETEM, Veszprém (HU)

(72) Inventors: Gábor JÁRVÁS, Veszprém (HU); Márton Géza SZIGETI, Veszprém (HU); Hajnalka JANKOVICS, Balatonalmádi (HU); Noémi KOVÁCS, Kemenessömjén (HU); Róbert FARSANG, Veszprém (HU)

(21) Appl. No.: 18/701,364

(22) PCT Filed: Oct. 14, 2022
(86) PCT No.: PCT/HU2022/050074

§ 371 Date: Apr. 15, 2024

(30) Foreign Application Priority Data
Oct. 14, 2021 (HU) P2100356

(51) Int. Cl. C12N 9/24; C12N 11/14; C12N 15/70

(52) U.S. Cl. CPC C12N 9/2402; C12N 11/14; C12N 15/70; C07K 2319/21; C12Y 302/01018; C12Y 302/01021; C12Y 302/01052

(57) ABSTRACT

The invention relates to a set of exoglycosidase enzymes which can be used in and/or are suitable for N-glycan sequencing, each one of the said exoglycosidases having different exoglycosidase activities specific for cleaving different terminal carbohydrates, and each one of the said exoglycosidases comprises a peptide tag. Said peptide tag, preferably HIS-tag may be used for potential immobilization to ensure the best accessibility to the active sites of the enzymes targeting automation purposes and special workflows.

The invention provides rapid enzymatic digestion performance both in aqueous phase and in immobilized form. Immobilized enzymes allow long term storage and ready to use pre-mixing.

The inventive immobilization approach opens up the possibility for automation and for meeting special experimental needs where the immobilization of the enzymes is key.

### FIELD OF THE INVENTION

The present inventors have designed pure, soluble, and functional exoglycosidase enzymes that can be produced in high yield and cost-effective way using bacterial expression systems.

The invention relates to a set of exoglycosidase enzymes which can be used in and/or are suitable for N-glycan sequencing, each one of the said exoglycosidases having different exoglycosidase activities specific for cleaving different terminal carbohydrates, and each one of the said exoglycosidases comprises a peptide tag. Said peptide tag, preferably HIS-tag may be used for potential immobilization to ensure the best accessibility to the active sites of the enzymes targeting automation purposes and special workflows.

The invention provides rapid enzymatic digestion performance both in aqueous phase and in immobilized form. Immobilized enzymes allow long term storage and ready to use pre-mixing.

The inventive immobilization approach opens up the possibility for automation and for meeting special experimental needs where the immobilization of the enzymes is key.

### BACKGROUND ART

Protein glycosylation is one of the most common post-translational modifications in eukaryotic cells, and it has been known that carbohydrate structures contain a huge amount of biological information. They have key role in cell-cell interactions, in signaling pathways and they are implicated in disease progression. [Gabius, H. J. The sugar code: Why glycans are so important, Biosystems 164 (2018) 102-111.] N- and O-glycosylation are important for proper folding, stability and the functionality of proteins [Vliegenthart, J. F. The complexity of glycoprotein-derived glycans, Proc Jpn Acad Ser B Phys Biol Sci 93(2) (2017) 64-86.]. Such glycan moieties are composed of monosaccharide units, where monomer sequence, linkage type and position mean the complexity and diversity. Therefore, the structural analysis of glycans is a challenging task, but has critical importance in the understanding of their biofunction.

Biologics has outstripped small molecules thanks to their outstanding specificity and efficacy. Most often, biotherapeutics are glycosylated monoclonal antibodies or fusion proteins. The carbohydrate moieties of biologics have a key role in biological activity, solubility and immunogenicity of therapeutics. Therefore, there is an essential need to characterize the glycan profile of glycoproteins.

Exoglycosidase digestion is a usual method for the characterization of glycans. Archer Hartmann et al. [Archer Hartmann et al. Microscale exoglycosidase processing and lectin capture of glycans with phospholipid assisted capillary electrophoresis separations, Anal Chem 83(7) (2011) 2740-7.] teach an in-capillary cleavage method of terminal glycan residues with exoglycosidases.

Yamagami, M. et al. also describe an in-capillary method wherein online exoglycosidase digestion was combined with a plug-plug kinetic mode of capillary electrophoresis (CE) for the analysis of glycoprotein-derived oligosaccharides. [Yamagami, M. et al. Plug-plug kinetic capillary electrophoresis for in-capillary exoglycosidase digestion as a profiling tool for the analysis of glycoprotein glycans, J Chromatogr A 1496 (2017) 157-162.]

Song T. et al. [Song T. et al. In-depth method for the characterization of glycosylation in manufactured recombinant monoclonal antibody drugs, Anal Chem 86(12) (2014) 5661-6.] describe a liquid chromatography-mass spectrometry (LC-MS) method to prepare N-glycan library based on eight commercial rMab drugs. The complete glycan structures were obtained through exoglycosidase sequencing.

Oligosaccharide sequencing by exoglycosidase digestion is one of the most commonly used techniques to determine the structure of complex glycans [Guttman, M. et al. Comparative glycoprofiling of HIV gp120 immunogens by capillary electrophoresis and MALDI mass spectrometry, Electrophoresis 36(11-12) (2015) 1305-13; Viradi, C. Analysis of cetuximab N-Glycosylation using multiple fractionation methods and capillary electrophoresis mass spectrometry, J Pharm Biomed Anal 180 (2020) 113035.].

Enzyme digestion provide accurate sequence and linkage information of oligosaccharide chains. Exoglycosidase enzymes have monosaccharide unit and linkage orientation (α vs β) specificity, so they can reveal not only the sequence of glycans but also their anomeric configuration [Gattu S. et al. Microscale Measurements of Michaelis-Menten Constants of Neuraminidase with Nanogel Capillary Electrophoresis for the Determination of the Sialic Acid Linkage, Anal Chem 89(1) (2017) 929-936, Lu, C. L. et al. Capillary Electrophoresis Separations of Glycans, Chem Rev 118(17) (2018) 7867-7885] [11, 21]. Glycan sequencing requires multiple separation of the reaction mixtures, where CE is frequently used thank to its low sample volume need and short separation time. The structural information can be derived from the peak shifts of the consecutive exoglycosidase treatments.

One of the limitations of enzymes for the industrial utilization is that the enzymes rapidly lose their activity, which can significantly increase the cost of analysis. Immobilization of the enzymes may avoid the efficiency decrease [Sheldon R. A., S. van Pelt, Enzyme immobilisation in biocatalysis: why, what and how, Chem. Soc. Rev. 42(15) (2013) 6223-6235.]. Furthermore, immobilization allow repeated use, as well as improve resistance to denaturation. Immobilization may enhance enzyme activity, however, this is not evident. Numerous immobilization techniques are reported for various applications [Krenkovi J., F. Foret, Immobilized microfluidic enzymatic reactors, Electrophoresis 25(21-22) (2004) 3550-63.].

Temporini et al. developed an on-line immobilized enzyme reactor technique coupled with HPLC-MS/MS method for the simultaneous characterization of glycan and peptide moieties in pronase-generated glycopeptides. [Temporini, C. et al. Pronase-Immobilized Enzyme Reactor: an Approach for Automation in Glycoprotein Analysis by LC/LC-ESI/MSn, Analytical Chemistry 79(1) (2007) 355-363.] The pronase enzyme was immobilized on an epoxy-silica monolithic material. The immobilization of pronase decreased the reaction time from 48 h to 40 min.

Krogh, T. N. et al. Protein Analysis Using Enzymes Immobilized to Paramagnetic Beads Analytical Biochemistry 274, (1999) 153-162 use chemical immobilization of trypsin as well as of exo- and endoglycosidases to paramagnetic beads to demonstrate a protein analysis method combining protein chemistry with enzymes. Immobilization is said to have resulted in high sensitivity, faster glycosidase digestion of glycopeptides, and reduced sample contamination. As an analysis method MALDI-MS has been used.

Immobilized-enzyme pipettes have been developed as an efficient tool for micro-volume chromatography analysis as well as an enzymatic micro-reactor fit in modern laboratory setting. In glycomic studies immobilization of enzymes such as PNGase F in pipette tip have previously been applied to rapidly analyze glycoproteins [Chen, J. et al. Solid phase extraction of N-linked glycopeptides using hydrazide tip, Anal Chem 85(22) (2013) 10670-4, Yamamoto, S. et al. A fast and convenient solid phase preparation method for releasing N-glycans from glycoproteins using trypsin- and peptide-N-glycosidase F (PNGase F)-impregnated polyacrylamide gels fabricated in a pipette tip, Journal of Pharmaceutical and Biomedical Analysis 179 (2020) 112995.]

Automation as well as speed of analysis, including development of high throughput techniques is a challenge continuously present in the field of glycan sequencing.

In a recent study, rapid and automated exoglycosidase digestion based carbohydrate sequencing of human immunoglobulin G (IgG) and Enbrel (etanercept) fusion protein N-glycans was described wherein the sample storage compartment of the CE instrument was used for reaction temperature control and the separation capillary as enzyme delivery device. [Szigeti, M and Guttman, A Automated N-Glycosylation Sequencing Of Biopharmaceuticals By Capillary Electrophoresis, Sci Rep 7(1) (2017) 11663; Guttman A, Szigeti M: Fast Glycan Sequencing Using a Fully Automated Carbohydrate Sequencer, Sciex, https://sciex.com/products/consumables/fast-glycan-analysis-and-labeling-for-the-pa-800-plus 2017-09-14] However, no immobilization was applied and the method consisted of a series of individual digestion and sequencing steps.

While glycan sequencing methodologies have developed significantly in the past few years there is still a need in the art to provide fast and robust sequencing methodologies based on immobilized exoglycanases.

The present inventors have provided a glycan sequencing system based on the use of a series of engineered tagged exoglycanases in particular immobilized via a peptide tag to a solid matrix support and a system for N-glycan sequencing.

### BRIEF DESCRIPTION OF THE INVENTION

1. The invention relates to a set of exoglycosidase enzymes which can be used in and/or are suitable for N-glycan sequencing, each one of the said exoglycosidases having different exoglycosidase activities specific for cleaving different terminal carbohydrates from non-reducing end of a glycan, and each one of the said exoglycosidases comprises a peptide tag for immobilization of said exoglycosidase on a matrix support, said peptide tag being present in a position of the exoglycosidase sequence wherein it does not affect exoglycosidase activity.

In alternative wording the set of exoglycosidase enzymes is a plurality (i.e. more than one) of exoglycosidase enzymes, or a series or a system of exoglycosidase enzymes.

The peptide tag is preferably a methyl chelating peptide tag, highly preferably a His-tag.

In a highly preferred embodiment the set of exoglycosidase enzymes comprises at least two enzymes selected from the following group of enzymes: a Neuraminidase, a Galactosidase and a Hexosaminidase.

Preferably, said set of exoglycosidases comprises at least
a Neuraminidase, preferably alpha Neuraminidase, having the activity for cleaving a terminal sialic acid from a glycan and wherein said peptide tag is engineered to the C-terminal of the Neuraminidase,
a β-Galactosidase, wherein said peptide tag is engineered to the N-terminal of the β-Galactosidase and
a Hexosaminidase, wherein said peptide tag is engineered to the C-terminal of the Hexosaminidase, and
wherein the level of Hexosaminidase is increased relative to Neuraminidase and beta-Galactosidase so as to have hexosaminidase activity sufficient to achieve a complete cleavage of the terminal carbohydrate during a pre-determined time-period defined for each exoglycosidases in the set.

In a preferred embodiment said exoglycosidases (preferably each of them) are immobilized on said matrix support.

In a preferred embodiment said set of exoglycosidases comprises more than one subsets of exoglycosidases, each subsets being different in at least one exoglycosidase from each other subset(s),
wherein a subset may comprise a single exoglycosidase,
within a subset each exoglycosidases having different exoglycosidase activities specific for cleaving different terminal carbohydrates from non-reducing end of a glycan,
wherein preferably in a subset each exoglycosidase is immobilized on the same matrix support.

In a preferred embodiment each of said exoglycosidases are immobilized on said matrix support, and preferably in a subset each exoglycosidase is immobilized on the same matrix support.

2. Preferably the invention relates to a set of exoglycosidases according to paragraph 1
wherein the level of said exoglycosidases in the set is adjusted so that each exoglycosidase have exoglycosidase activity sufficient to achieve a complete cleavage of the terminal carbohydrate during a pre-determined temperature range and time-period defined for each exoglycosidases in the set.

Preferably the pre-determined time-period is at most 1.0 h, preferably at most 40 minutes, preferably at most half hour or 30 minutes, and wherein preferably the temperature range is 37° C. to 60° C. Preferably the temperature range is 37° C. to 60° C. and the time-range is 5.0 min to 30 min.

Highly preferably the pre-determined time-period is at most half hour or 30 minutes, and wherein the temperature range is 37° C. to 60° C.

Preferably the level of said exoglycosidases are coordinated, i.e. their level is adjusted to each other to proved a comparable activity or an activity each capable of carrying out complete digestion within a time period or time range.

3. In a preferred embodiment the invention relates to the set of exoglycosidases according to any one of paragraphs 1 to 2, wherein
said set of exoglycosidases comprises an exoglycosidase having the activity for cleaving a terminal carbohydrate from N-glycans.

Preferably, said set of exoglycosidases comprises at least Neuraminidase, preferably alpha Neuraminidase, having the activity for cleaving a terminal sialic acid from a glycan and at least one further exoglycosidase, wherein preferably said peptide tag is engineered to the C-terminal of the neuraminidase.

4. In a preferred embodiment the invention relates to the set of exoglycosidases according to paragraph 1, 2, 3, preferably 3, wherein said at least one further exoglycosidase comprises at least a β-Galactosidase wherein preferably said peptide tag is engineered to the N-terminal of the β-Galactosidase.

In a preferred embodiment said at least one further exoglycosidase is selected from the group consisting of β-Galactosidase and Hexosaminidase.

In a preferred set of exoglycosidases the peptide tag is a His-tag and the matrix support is a transition metal-comprising matrix support, said transition metal more preferably being selected from Cu²⁺, Ni²⁺, Zn²⁺, and Co²⁺.

5. In a preferred embodiment the invention relates to the set of exoglycosidases according to any one of paragraphs 3 to 4, wherein in
said set of exoglycosidases the at least one further exoglycosidase comprises at least a Hexosaminidase, wherein preferably said peptide tag is engineered to the C-terminal of the Hexosaminidase,
said set of exoglycosidases the at least one further exoglycosidase comprises at least a Hexosaminidase, wherein preferably said peptide tag is engineered to the C-terminal of the Hexosaminidase,

6. In a preferred embodiment the invention relates to the set of exoglycosidases according to paragraph 5 wherein the level of Hexosaminidase is increased relative to Neuraminidase and beta-Galactosidase so as to have hexosaminidase activity sufficient to achieve a complete cleavage of the terminal carbohydrate during a pre-determined time-period defined for each exoglycosidases in the set. Thus, the levels of the enzymes are coordinated and thus adjusted to complete the cleavage reaction in a pre-determined time-period essentially during the same time-period.

7. In a preferred embodiment the invention relates to the set of exoglycosidases according to any one of paragraphs 1 to 6, wherein said peptide tag is a metal-chelating peptide tag and the matrix support is a metal-comprising matrix support.

In a particular embodiment, the peptide tag is selected from the group consisting of a metal-chelating peptide tag, an epitope peptide tag, a substrate peptide tag, a ligand peptide tag, a modified peptide tag.

Preferably the matrix support comprises binding moieties for binding of the peptide tag, in the a particular embodiment the matrix support comprises metal ion, antibody or any binding molecule having an epitope binding site, a substrate binding molecule, a receptor binding the ligand, a binding molecule binding the modified peptide tag.

Optionally the modified peptide tag is modified for allowing covalent binding to the matrix support.

8. In a preferred embodiment the invention relates to the set of exoglycosidases according to any of paragraphs 1 to 7, said exoglycosidases are immobilized on a matrix support; preferably the exoglycosidases mixed together are dialyzed together (co-dialyzed) to reduce negative salt effect.

9. In a preferred embodiment the invention relates to the set of exoglycosidases according to any one of paragraphs 1 to 8, wherein each of said exoglycosidases are immobilized on a matrix support, in particular by a means according to paragraph 7.

10. In a preferred embodiment the invention relates to the set of exoglycosidases according to any one of paragraphs 1 to 9, wherein more than one exoglycosidases are immobilized on the same matrix support.

11. In a preferred embodiment the invention relates to the set of exoglycosidases according to any one of paragraphs 1 to 10 comprising more than one subsets of exoglycosidases, each subsets being different in at least one exoglycosidase from each other subset(s), wherein a subset may comprise a single exoglycosidase,
within a subset each exoglycosidases having different exoglycosidase activities specific for cleaving different terminal carbohydrates from non-reducing end of a glycan.

12. In a preferred embodiment the invention relates to the set of exoglycosidases according to paragraph 11, wherein within a subset each exoglycosidase is immobilized on the same matrix support.

13. In a further aspect the invention relates to a use of a set of exoglycosidases according to any one of paragraphs 1 to 12 for N-glycan sequencing. Preferably, glycan cleavage reactions with multiple subsets of exoglycosidases are carried out simultaneously and each of the reaction mixtures are analyzed to obtain glycan sequence information. Preferably the exoglycosidases are defined in any of paragraphs 1 to 12, in particular 1, preferably the preferred options therein, or any of paragraphs 6 to 12, in particular or any of paragraphs 7 to 12 or 8 to 12.

14. In a preferred embodiment the invention relates to the use of a set of exoglycosidases according to paragraph 13 wherein the result (reaction mixture) of the glycan cleavage reactions (digestion) is analyzed to obtain glycan sequence information. Preferably the result (resulting reaction mixture, i.e. the cleaved glycan product) is analyzed by a separation method selected from the group consisting of HPLC, UPLC and capillary electrophoresis, preferably with fluorescence detection.

Preferably analysis is carried out by capillary electrophoresis, preferably with fluorescence detection. 15. In a preferred embodiment the invention relates to the use of a set of exoglycosidases according to any of paragraphs 13 to 14 wherein glycan cleavage reactions (for N-glycan sequencing) with multiple subsets of exoglycosidases as defined in any of paragraphs 11 to 12 are carried out simultaneously (i.e. in parallel) and each of the reaction mixtures are analyzed to obtain glycan sequence information.

In a preferred embodiment in this analysis the separation result (e.g. chromatogram(s) or electropherogram(s)) is carried out to obtain said sequence. In case multiple cleavage reactions are carried out the results of these reactions are compared and assembled to obtain said sequence information.

In an exemplary embodiment sequential cleavage reactions are carried out and in each reaction mixture the sugar moiety is identified.

In a preferred embodiment sequencing is carried out with the set of exoglycosidases and with subsets thereof wherein each subsets being different in at least one exoglycosidase from each other subset(s),
wherein a subset may comprise a single exoglycosidase,
within a subset each exoglycosidases having different exoglycosidase activities specific for cleaving
different terminal carbohydrates from non-reducing end of a glycan,
and each of the reaction mixtures are analyzed to obtain glycan sequence information;
wherein preferably in a subset each exoglycosidase is immobilized on the same matrix support.

In another exemplary embodiment the results of the cleavage reaction with various enzyme mixtures are analyzed and the sugar composition is assessed (measured or identified) whereby, once these composition data are compared and assembled, the glycan sequence is determined.

As to an example for assessing sugar composition see Table 1 and the related description.

16. In a further aspect the invention relates to a method for the preparation of a set of exoglycosidases according to any of paragraphs 1 to 12, wherein
nucleic acid sequences encoding the exoglycosidases are provided,
nucleic acid sequences encoding the peptide tag are inserted into said nucleic acid sequences encoding the exoglycosidases (e.g. by molecular cloning), so that the peptide tag, once expressed, is present in a position of the exoglycosidase sequence wherein it does not affect exoglycosidase activity,
each exoglycosidase is expressed in an expression system, preferably in a bacterial expression system,
each exoglycosidase is isolated (highly preferably using the engineered His peptide tag on a metal chelate column),
   preferably the isolation comprises a step wherein the exoglycosidase is bound to a chromatography matrix via the peptide tag,
the levels of the exoglycosidases are adjusted so that each exoglycosidase have exoglycosidase activity sufficient to achieve a complete cleavage of the terminal carbohydrate in a pre-determined temperature range and during a pre-determined time-period defined for each exoglycosidases in the set.
   Preferably the pre-determined time-period is at most 1.0 h, preferably at most 40 minutes, preferably at most half hour or 30 minutes, and wherein preferably the temperature range is 37° C. to 60° C. Preferably the temperature range is 37° C. to 60° C. and the time-range is 5.0 min to 30 min.

Preferably the level of said exoglycosidases are coordinated, i.e. their level is adjusted to each other to proved a comparable activity or an activity each capable of carrying out complete digestion within a time period or time range.

Preferably the matrix support comprises binding moieties for binding of the peptide tag, in the a particular embodiment the matrix support comprises metal ion, antibody or any binding molecule having an epitope binding site, a substrate binding molecule, a receptor binding the ligand, a binding molecule binding the modified peptide tag.

Optionally the modified peptide tag is modified for allowing covalent binding to the matrix support.

17. In a preferred embodiment the invention relates to the method for the preparation of a set of exoglycosidases according to paragraph 16 wherein at least a part of the exoglycosidases are mixed together to form a single cleavage reaction mixture, preferably a subset of exoglycosidases are mixed together as defined in any of paragraphs 11 to 12.

18. In a preferred embodiment the invention relates to the method for the preparation of a set of exoglycosidases according to paragraph 17 wherein the exoglycosidases mixed together are dialyzed together (co-dialyzed) to reduce negative salt effect on signal strength during capillary electrophoretic analysis.

In a preferred embodiment co-dialysis is highly preferred for CE and/or HPLC separation analysis.

19. In a preferred embodiment the invention relates to the method for the preparation of a set of exoglycosidases according to any of paragraphs 16 to 18 wherein the exoglycosidases are immobilized to a matrix support via the peptide tag. Preferably the peptide tag is a tag as defined in claim 16.

20. In a preferred embodiment the invention relates to the method for the preparation of a set of exoglycosidases according to paragraph 19 wherein
more than one subsets of exoglycosidases are mixed together and co-dialyzed,
each subsets being different in at least one exoglycosidase from each other subset(s), wherein a subset may comprise a single exoglycosidase,
within a subset each exoglycosidases having different exoglycosidase activities specific for cleaving different terminal carbohydrates from non-reducing end of a glycan.
and within a subset each exoglycosidase is immobilized on the same matrix support.

Preferably the peptide tag is a tag as defined in claim 16.

In a highly preferred embodiment the peptide tag is a metal chelating tag, in particular a His-tag.

21. A kit for preparing an immobilized set of exoglycosidases as defined in any of paragraphs 11 to 12, said kit comprising
a set of soluble exoglycosidases as defined in paragraphs 1 to 10 wherein said exoglycosidases are present in soluble form in buffer solution(s) as a stock exoglycosidase solution or set of stock exoglycosidase solutions,
a matrix support functionalized to be able for binding to or by the peptide tag engineered into the exoglycosidases,
optionally buffers and reagents to carry out immobilization, buffers for washing the matrix support before or after immobilization and storage buffers said storage buffers comprising preferably a stabilizing agent, preferably glycerin.

### Abbreviations

APTS: 8-Aminopyrene-1,3,6-trisulfonic acid trisodium salt; AmAc: ammonium acetate; PNGase F: Peptide-N-glycosidase F; CE: capillary electrophoresis; MD: maltodextrin; NANase: neuraminidase-TEV-6HIS enzyme; GALase: 6HIS-TEV-β-galactosidase enzyme; HEXase: hexosaminidase-TEV-6HIS enzyme; hIgG1: human immunoglobulin G1.

### Definitions

“Exoglycosidase enzymes” (or exoglycosidases in short) are glycoside hydrolase (EC 3.2.1) enzyme which breaks (or cleaves) the glycosidic bonds at the terminal residue.

Exoglycosidases include, among others, enzymes with the following specificity:
neuraminidase, in particular alpha neuraminidase, galactosidase, in particular beta-galactosidase, glucosaminidase, in particular N-acetylglucosaminidase, more particularly beta N-acetylglucosaminidase, mannosidase, in particular alpha- and/or beta-mannosidase, fucosidase, in particular alpha-fucosidase etc.

Without limitation exoglycosidases are described in the art [Kobata A. (2013). Exo- and endoglycosidases revisited. Proceedings of the Japan Academy. Series B, Physical and biological sciences, 89(3), 97-117.]

“N-glycan sequencing” is a process wherein exoglycosidase enzymes (exoglycosidases) remove terminal carbohydrates from the non-reducing end of a glycan, but do not cleave internal bonds between carbohydrates and thereby, by using positionally specific exoglycosidases, the removed glycan residues can be identified, preferably by linkage as well as sugar,

A “peptide tag” as defined herein is a peptide the coding sequence of which is inserted into or added to the coding sequence of a protein, here an exoglycosidase enzyme, whereby the expressed protein also comprises the peptide tag, and wherein said peptide tag is capable of binding to a binding moiety carried by a matrix (matrix support) to form, in a terminology, a resin or a matrix support with a binding moiety. Preferably the “peptide tag” is used for immobilization to the matrix support. Preferably the “peptide tag” is used for isolating or purifying the protein wherein the matrix support is a chromatography matrix. The matrix support comprises an inert carrying material and a binding moiety capable of binding or binding to the peptide tag to form a resin. Optionally the carrying material and the binding moiety are linked by a linker (moiety). In case of metal-chelating tags like the His-tag the resin is a metal-containing resin.

Peptide tags suitable for immobilization and preferably also purification include, among others, epitope tags bound by antibodies or other epitope binding molecules like FLAG-tag, HA-tag, Myc-tag, NE-tag, or a tag of a ligand-binding protein pair like a Calmodulin tag, a peptide bound by calmodulin or an SBP-tag or a Strep-tag, peptides which bind to streptavidin or modified version thereof, or a modified peptide tag, e.g. a peptide tag useful for covalent binding, or a metal-chelating tag like a His-tag, preferably comprising or consisting of 5-10 His amino acid, preferably a 6His-tag having 6 His amino acid.

A “set” of enzymes as used herein is a group or collection of enzymes, considered as an entity unto itself. Thus, in a preferred embodiment the members of a set are capable of working together and/or are useful to achieve a result to which each member of the set is utilized. A set is composed of similar enzyme (set of enzymes having a common property). A set is a kit, however, in a kit consists of parts which may differ from each other as their basic nature (e.g. a kit may comprise enzyme, buffer, some device, resin, etc.).

A “subset” is a set which is part of a set; i.e. it may be identical with the set or (in a narrower meaning) a smaller part of a set which comprises a lesser member than the (whole) set itself.

The meaning of “level” of the exoglycanase enzymes in a set or subset of enzymes may refer to the amount or concentration of the individual exoglycanases with different substrate (carbohydrate linkage) specificities to coordinate their activity, ideally to arrive at full cleavage of the terminal sugar moiety in the digestion reaction for each exoglycosidase.

A “group” (or “moiety”) is used herein as a part of a molecule or a complex which can be derived in principle by re-moving another part or any other part, like a hydrogen atom (wherein the moiety is usually a negative charged ion) or a metal ion (wherein the moiety may be a chelating agent) or an organic part of a complex (wherein the moiety may be a metal ion).

A “chelate” comprises at least two “coordinate bond” or “dative bond”, i.e. a two, center, two, electron bond in which the two electrons derive from the same atom, typically a non¬binding electron pair is coordinated to a metal ion. Such a bond is marked in an undulating line in the formulae.

The singular forms “a”, “an” and “the”, or at least “a”, “an”, include plural reference unless the context clearly dictates otherwise.

The term “comprises” or “comprising” or “including” are to be construed here as having a non-exhaustive meaning and allow the addition or involvement of further features or method steps or components to anything which comprises the listed features or method steps or components. “Comprising” can be substituted by “including” if the practice of a given language variant so requires or can be limited to “consisting essentially of” if other members or components are not essential to reduce the invention to practice.

### BRIEF DESCRIPTION OF THE FIGURES

FIG. 1.—Gene design for NANase (left), GALase (middle) and HEXase (right). For protein expression the genes were incorporated into an engineered pET23b plasmid between the NdeI and XhoI sites resulting in the pET23b-NdeI-neuraminidase-TEV-6HIS-XhoI (“NANase”) and NdeI-hexosaminidase-SacI-TEV-6HIS-XhoI constructions (“HEXase”); whereas for expression of β-galactosidase fusion of an N-terminal 6HIS-TEV tag to its functional part using an engineered pET17b-based plasmid [Materials and methods] was applied (“GALase”).

FIG. 2.—N-glycan sequencing of hIgG1 glycoprotein sample using a 37° C. overnight digestion applying enzymes individually to the sample (Panel A) and applying the enzymes pre-mixed and co-dialyzed (Panel B). Efficiency of the produced co-dialyzed enzyme mixtures (in 50% glycerol after 10× dilution) has been compared to the mixture of the individually used and glycerol-free enzymes. Separation conditions: 50 cm total (40 cm effective) length BFS capillary, NCHO separation gel buffer, 20° C. capillary and sample temperature, and pressure injection using 5.0 psi for 5.0 s.

FIG. 3.—Exoglycosidase digestion of the glycans of hIgG1 sample using immobilized enzyme mixtures. Enzymes were immobilized on PhyNexus microcolumns. Separation conditions were the same as in FIG. 2. By increasing the concentration of the HEXase to 0.6 μM (4×), complete reaction was obtained in 30 min using immobilized enzymes.

FIG. 4.—N-glycan sequencing of Daratumumab mAb glycans (panel A), human serum glycans (panel B) and Synagis mAb glycans (panel C) with the premixed and co-dialyzed enzymes in aqueous phase and in immobilized form using the temperature gradient method for 30 min. Separation conditions were the same as in FIG. 2. In all cases, complete exoglycosidase digestion was obtained in the given timeframe.

### DETAILED DESCRIPTION OF THE INVENTION

In the present invention sets of exoglycosidases are provided for use in glycan sequencing, wherein a set of exoglycosidase comprises two or more exoglycosidases,
each one of said exoglycosidases having different exoglycosidase activities specific for cleaving different terminal carbohydrates from non-reducing end of a glycan, and
each one of said exoglycosidases a peptide tag for immobilization of said exoglycosidase on a matrix support,
   said peptide tag being present in a position of the exoglycosidase sequence wherein it does not affect exoglycosidase activity.

In a particular embodiment the present inventors demonstrate the production and application of peptide tagged, highly preferably His-tagged or 6HIS tagged exoglycosidase enzymes such as Neuraminidase, β-Galactosidase and Hexosaminidase in order to rapidly perform the N-glycan sequencing of glycoproteins. The enzymes are capable of high-performance digestion in aqueous phase and in immobilized forms for e.g., automated solutions.

His tagging is known in the art in general, even in case of beta-glucosidase [Zhou Y et al: Synchronized purification and immobilization of His-tagged beta-glucosidase via Fe3O4/PMG core/shell magnetic nanoparticles. Sei Rep 7, 41741 (2017)], beta galactosidase-1 [R&D Systems: Recombinant human beta-Galactosidase-1, His-tagged (cat. &6464-GH)] or hexosaminidase [R&D Systems: Recombinant human hexosaminidase A/HEXA, His-tagged (cat. #6237-GH)].

In further preferred embodiments, the enzymes were premixed to form subset(s) of exoglycosidases whereby, upon digestion, the reaction mixture as a result of the activity of multiple exoglycosidases may be obtained.

In a highly preferred embodiment all the applied enzyme mixtures were co-dialyzed together in order to reduce the negative salt effect on signal strength during capillary electrophoretic analysis. In an embodiment co-dialization was carried out to the very same concentration.

In a further preferred embodiment the mixtures as subsets are used for simultaneous reactions of glycan digestion (terminal carbohydrate cleavage.

It is particularly important to select the proper immobilization technique to achieve the most efficient enzyme reaction because of immobilization influences the catalytic activity of enzymes. In the present invention peptide tags are used for immobilization of the exoglycosidases. Peptide tags can be engineered genetically to any part of the molecule and thus are universally applicable in any exoglycosidase. This is important in the present set of exoglycosidases which are to be handled analogously, i.e. the same way. Each recombinant exoglycosidase has to be designed to some extent to introduce the peptide tag into a site wherein it does not interference enzyme activity.

In a preferred embodiment tags are used which are able to bind a metal-containing resin comprising a matrix (or matrix support and metal ions immobilized thereon, wherein immobilization comprises in particular the use of chelate ligands.

Metal ions immobilized on the metal-containing resins are e.g. transition metals e.g. selected from the group consisting of Cu²⁺, Ni²⁺, Zn²⁺, and Co²⁺, preferably Ni²⁺. The HIS-tag, for example, has a high affinity for these metal ions and binds strongly to these resins.

It is an advantage of peptide tags that they are also useful in the purification of the proteins. Thereby the preparation method of the inventive exoglycosidase sets is simple and straightforward as the same tag can be applied of isolation (or purification) and immobilization.

During isolation of a protein of interest from e.g. cell lysates, e.g. bacterial lysates, e.g. in case of His tags, most other proteins in the lysate will not bind to the resin, or bind only weakly. In an example, a low concentration of imidazole is added to both binding and wash buffers to interfere with the weak binding of other proteins and to elute any proteins that weakly bind. The tagged e.g. His-tagged protein can be then eluted with a higher concentration of an agent disrupting the tag-matrix binding, e.g. in case of His tags imidazole.

The same method can be used for regeneration of the matrix support. The tagged proteins are immobilized to the matrix support form a resin which can be used for multiple rounds of cleavage reactions and are stable. However, they can be regenerated by removing and re-adding a fresh preparation of tagged exoglycanases thereby regenerating the resin.

Matrix support may be made of any suitable matrix, e.g. without limitation agarose, e.g. 4% agarose or 6% highly cross-linked agarose.

As to exemplary further matrices, such support include but not limited to inorganic materials such as metal oxides, minerals, carbon materials, organic materials biopolymers such as cellulose, chitosan, agarose and synthetic polymers with without imprints.

In particular embodiments, wi, e.g. nitrilotriacetic acid (NTA) or iminodiacetic acid (IDA).

Combinations of these three variables (matrix, ligand, and ion) provide a collection of resins.

Other tags are also applicable for example engineered tags which are then chemically modified later. Such tags are e.g. aldehyded tags. For example formylglycine generating enzyme (FGE) can transform the cysteine from a conversed 6-amino-acid sequence CXPXR into formylglycine with an aldehyde group (also termed as “aldehyde tag”). [Wang F, Li R, Jian H, Huang Z, Wang Y, Guo Z, Gao R. Design and Construction of an Effective Expression System with Aldehyde Tag for Site-Specific Enzyme Immobilization. Catalysts. 2020; 10(4):410].

Other types of tags useful for immobilization and binding are well known and listed in the definition or in

Exoglycosidase digestion of glycans typically performed overnight and 37° C. incubation. In the present invention a fast and robust exoglycosidase sequential method by CE has been developed. A preferred embodiment of this method was accomplished with fluorescent detection of aminopyrene trisulfonate (APTS) labeled N-glycans. The exoglycosidase enzyme digestion performed rapidly by adjusting the pH of buffer, applying a temperature gradient, and immobilizing the enzymes.

In a particular embodiment the exoglycanase enzymes in a set or subset of enzymes are used together by adjusting the level (i.e. amount or concentration) of the individual exoglycanases with different substrate (carbohydrate linkage) specificities to coordinate their activity, ideally to arrive at full cleavage of the terminal sugar moiety in the digestion reaction for each exoglycosidase.

It is also important to engineer the peptide tag to a position of the exoglycosidase which does not disturb (impair) or reduce the exoglycosidase activity and also allows binding to the matrix support. This can be done for any exoglycosidase the structure of which is known to a sufficient extent to assess the catalytic site and part of the molecule which is sufficiently exposed to be suitable for binding, via the tag, to a matrix. A homology model or even sequence analysis to assign function and structural information to parts of the molecule may well be suitable for this purpose if 3D structure from measurements is not known.

A useful part of this engineering method is to remove parts of the exoglycosidase which are unnecessary for activity, i.e. to “simplify” the molecule. As examples the following enzymes have been used herein:

Beta-Galactosidase—the inventors have used bacterial (here: Streptococcus pneumoniae) enzyme so as to achieve a high level production in bacterial expression system, e.g. E. coli. The enzyme was a cell surface protein, which makes it useful for extracellular use. From 17 subunits the first five (subunits 1 to 5) are responsible for catalytic activity, these five subunits have been cloned only. Based on 3D, i.e. X-ray crystal structure (PDB: 4cu6) the portion having beta galactosidase activity (137-985 aminosavak) it was found that a peptide tag (e.g. 6His) added to the N-terminal would be suitable for immobilization wherein the active center would be well available for exoglycanase reaction. The structure, origin an function of the full enzyme (2233 amino acids) are described by Singh A. K. et al. [Singh A. K. et al. Unravelling the multiple functions of the architecturally intricate Streptococcus pneumoniae beta-galactosidase, BgaA PLoS Pathog. 10:e1004364-e1004364(2014)]

Design of Neuraminidase and Hexoseaminidase—The sequences of these enzymes are also publicly available. In the present approach the export signal and the peptide anchoring the protein to the cell wall were left out from the construct.

The polypeptide chain and structure of the (bacterial) Neuraminidase used herein as a start of the design is disclosed by Christensen S and Egebjerg J. [Christensen S and Egebjerg J. Cloning, expression and characterization of a sialidase gene from Arthrobacter ureafaciens. Biotechnol Appl Biochem. 2005 June; 41(Pt 3):225-31. doi: 10.1042/BA20040144. PMID: 15461582.]

For 3D structure a homology model is available in the SWISS-MODEL repository (Q5W7Q2 (Q5W7Q2_FLASK) Flavobacterium sp (strain K172), https://swissmodel.expasy.org/repository/uniprot/Q5W7Q2?template=2bf6.1.A&range=69-422.

Based on the homology model 3D structure the catalytic domain of the enzyme can be found at the N-terminal of the enzyme, however, the C-terminal domain also has role in the activity.

[Iwamori M et al. Involvement of the C-terminal tail of Arthrobacter ureafaciens sialidase isoenzyme M in cleavage of the internal sialic acid of ganglioside GM1. J Biochem. 2005 September; 138(3):327-34. doi: 10.1093/jb/mvi126. PMID: 16169883.]. The 6His tag (and the TEV-protease cleavage site) has been engineer to a sited near to the C-terminal of the molecule, to allow that the active center of the enzyme may be available for substrate after immobilization. Surprisingly the construct provided an active protein.

Hexoseaminidase—In case of Hexoseaminidase a crystal structure of the catalytic domains of the enzyme from Streptococcus pneumoniae was available only and not about the whole molecule. The structural information is available [Pluvinage B, et al. Conformational analysis of StrH, the surface-attached exo-β-D-N-acetylglucosaminidase from Streptococcus pneumoniae. J Mol Biol. 2013 Jan. 23; 425(2):334-49. doi: 10.1016/j.jmb.2012.11.005. Epub 2012 Nov. 12. PMID: 23154168.]. In the enzymes re-designed by the present inventors as spacers the G5 domains were maintained to provide a better availability of the catalytic domains for the substrates after immobilization. The 6His tag and the TEV enzyme cleavage site was designed to the C-terminal portion of the molecule.

While a specific and particular design of the exoglycosidases may not be obvious in advance based on the art, the skilled person will understand that some solutions can be achieved for any exoglycosidase as there will be a site in the enzyme which is suitable for a peptide tag being sufficiently exposed and located far enough from the catalytic domain.

### Results and Discussion

### Gene Design and Construction

In the present invention exoglycosidase genes are tailored to comprise a peptide tag for immobilization.

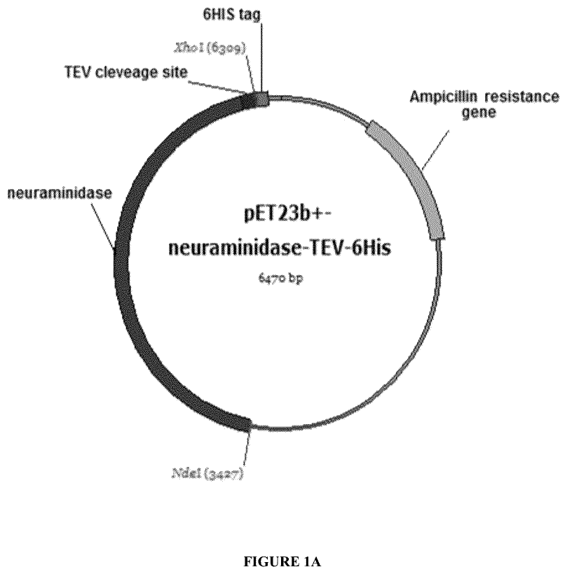

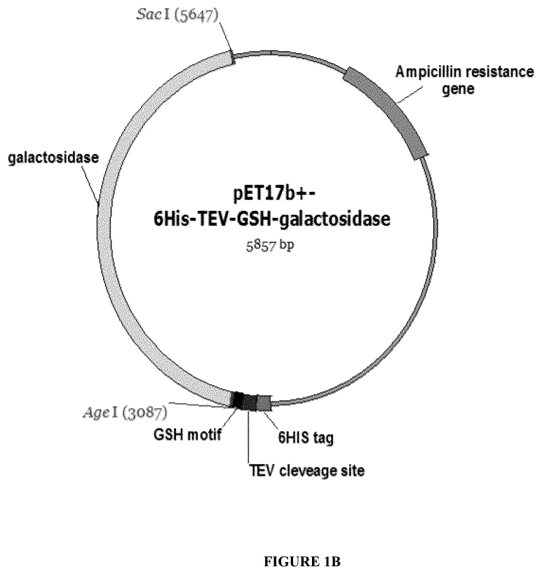

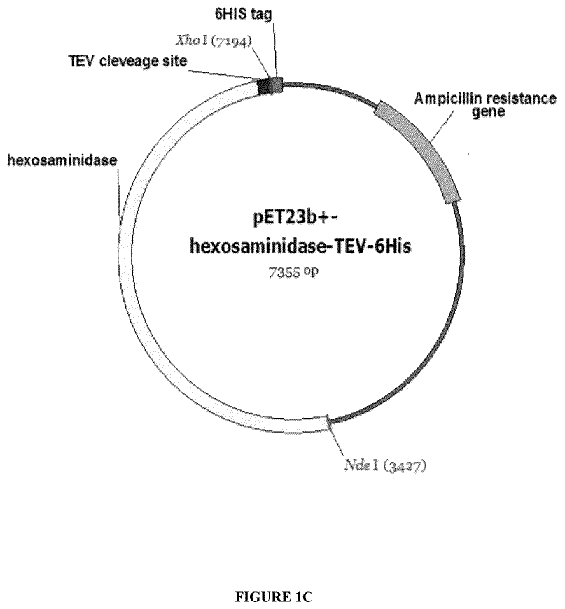

Genes of neuraminidase (EC 3.2.1.18), hexosaminidase (EC 3.2.1.52) and β-galactosidase (EC 3.2.1.23) exoglycosidases were designed to enable the expressed proteins to immobilize by their HIS-tag along the principles describe above. Considering the localization of the active center, the 6HIS tag, followed by a TEV protease cleavage site was fused to the C-terminal end of the enzymatically functional part of neuraminidase and hexosaminidase proteins. For protein expression the genes were incorporated into an engineered pET23b plasmid between the NdeI and XhoI sites resulting in the pET23b-NdeI-neuraminidase-TEV-6HIS-XhoI and NdeI-hexosaminidase-SacI-TEV-6HIS-XhoI constructions, respectively (FIG. 1). The proteins to be expressed are called herein as “NANase” and “HEXase”. In the case of β-galactosidase similar structural considerations led to the fusion of an N-terminal 6HIS-TEV tag to its functional part using an engineered pET17b-based plasmid [PNGaseF cikk hiv] for the expression of “GALase”.

Protein Expression in E. coli BL21 Strains

The six cysteins of NANase form three disulfide bridges at proper folding by oxidation, which is not supported in the reducing environment of the bacterial cytosol. Engineered E. coli cells, such as SHuffle T7 Express may support appropriate disulfide bridge formation resulting in soluble and catalytically active enzymes. Our attempt to express NANase in the right form was highly successful resulting in about 40 mg pure protein per liter culture. HEXase contains only a single Cys. SHuffle T7 Express may support right folding of proteins by preventing the formation of intermolecular S—S bonds, resulting in soluble and catalytically active enzyme. For HEXase in the properly folded form, 35 mg pure protein per liter culture was achieved. Since SHuffle usually produces significantly lower amount of cell mass, induced cultures were harvested at higher centrifugal force (10,000 g). Since GALase does not contain cysteine, expression of the enzyme was attempted in four different BL21 (DE3) strain and BL21 (DE3) Codon+RIL was found to be the most effective.

Purification of recombinant NANase, HEXase and GALase have been carried out, after sample preparation steps, on a HiTrap Chelating Ni-affinity column wherein elution was carried out by buffered imidazole solution.

Purity of the proteins in the eluted fractions were assessed by SDS-PAGE and protein concentration was calculated.

### Long Term Storage and Overnight Reference Separations

The optimal concentrations of each enzyme for complete APTS labeled glycan digestion were determined by preliminary experiments in aqueous phase one by one without buffering of the denatured protein sample. It is important to note that in case of the exoglycosidase enzyme digestion for N-glycan sequencing, only complete digestion is acceptable for uncompromised peak identification. The optimal concentration resulted to be 0.15 μM for each enzyme which were then pre-mixed and co-dialyzed in a buffer of 20 mM Tris-HCl, 50 mM NaCl, pH=7.5 in order to contain the same amount of salt having twenty times of enzyme concentration. Then, the mixtures were diluted 1:1 with glycerol and stored at −20° C. upon use. This approach resulted 1) long term storage of the enzymes that are ready to use after dilution, 2) negligible salt effect during injection for CE analysis and 3) high performance enzymatic reaction even in the presence of the glycerol in the storage mixture since the 10× dilution of the mixture prior use is sufficient to reduce its inhibition effect to a negligible level.

Three different compositions of solutions were prepared such as A) NANase only (0.3 mg/mL)—Mixture 1; B) NANase (0.3 mg/mL) and GALase (0.3 mg/mL)—Mixture 2 and C) NANase (0.3 mg/mL) and GALase (0.3 mg/mL) and HEXase (0.42 mg/ml)—Mixture 3. The concentration of HEXase was somewhat higher than that of the other two enzymes.

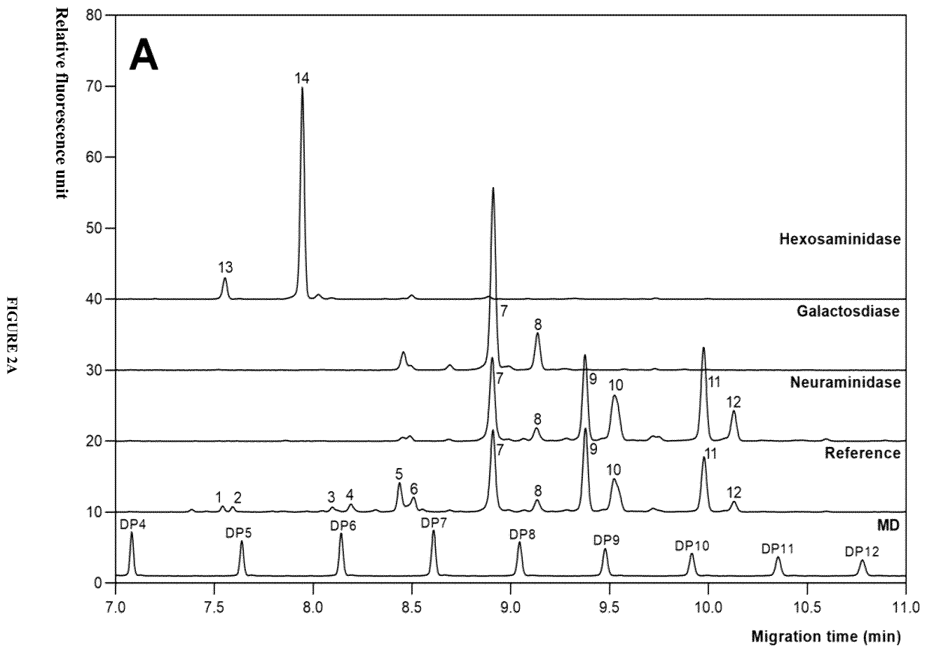

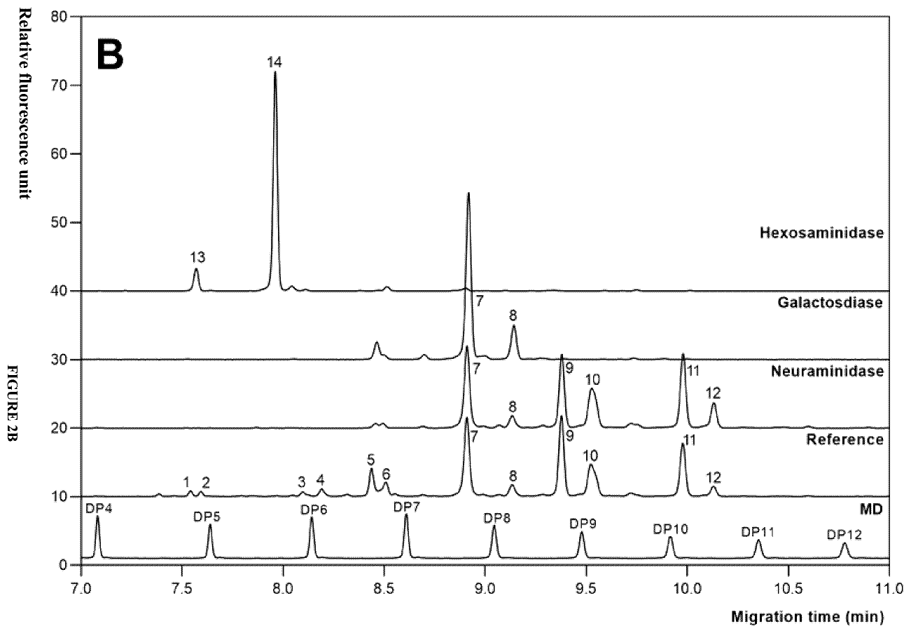

The efficiency of the produced co-dialyzed enzyme mixtures (in 50% glycerol after 10× dilution) has been compared to the mixture of the individually used and glycerol-free enzymes using the very same enzyme concentrations at 37° C. overnight (FIG. 2). Panel A shows N-glycan sequencing of exemplary hIgG1 glycoprotein sample using a 37° C. overnight digestion applying enzymes individually to the sample and Panel B shows the experiment wherein pre-mixed and co-dialyzed enzymes were applied.

The results shown in FIG. 2 and Table 1 show that there is no significant difference in % areas between the standard and the co-dialyzed (stored in 1:1 glycerol) approach. Thus, the established storage and pre-mixing method can be used for further use and examination.

TABLE 1
The resulted % area values during the N-glycan sequencing
of hIgG1 sample comparing the standard, individually
used approach to the premixed, co-dialyzed solution.

% area

| | | | |
|---|---|---|---|
| Peak no. | Structure | Standard | Co-dialyzed |

Neuraminidase digestion

| | | | |
|---|---|---|---|
| 7 | FA2 | 22.43 | 22.47 |
| 8 | FA2B | 3.41 | 3.63 |
| 9 | FA2[6]G1 | 21.94 | 21.68 |
| 10 | FA2[3]G1 | 18.47 | 18.58 |
| 11 | FA2G2 | 25.65 | 25.97 |
| 12 | FA2BG2 | 8.09 | 7.67 |

Galactosidase digestion digestion

| | | | |
|---|---|---|---|
| 7 | FA2 | 83.45 | 83.24 |
| 8 | FA2B | 16.55 | 16.76 |

Hexosaminidase digestion digestion

| | | | |
|---|---|---|---|
| 13 | M3 | 9.60 | 9.48 |
| 14 | FM3 | 90.40 | 90.52 |

### Rapid Aqueous Phase and Immobilized Exoglycosidase Digestion

All enzymatic reaction using the three 6HIS tagged enzyme mixtures were done using a temperature gradient method.

Also, to maximize the overall reaction speed of enzymes working in parallel, the sample were buffered using 10 mM ammonium acetate at pH=4.5. Because of the properties of the 6HIS tagged enzymes, the temperature gradient steps were further optimized and reduced to 30 min using 1) 37° C. for 5.0 min 2) heat up to 50° C. in 3.0 min 3) 50° C. for 12 min 4) heat up to 60° C. in 3.0 min and 5) 60° C. for 7.0 min resulting complete N-glycan sequencing in an excellent timeframe.

The experiments then were repeated with the same parameters using immobilized enzymes on Ni-IMAC resin. In the examples PhyNexus microcolumns were used for this purpose. As small as 40 μL bed volume with 1 ml pipette tip volume was sufficient for the analysis. In this method bidirectional flow can be applied which improves the reaction efficiency.

Pipetting and thereby the method can be automated with a controller software.

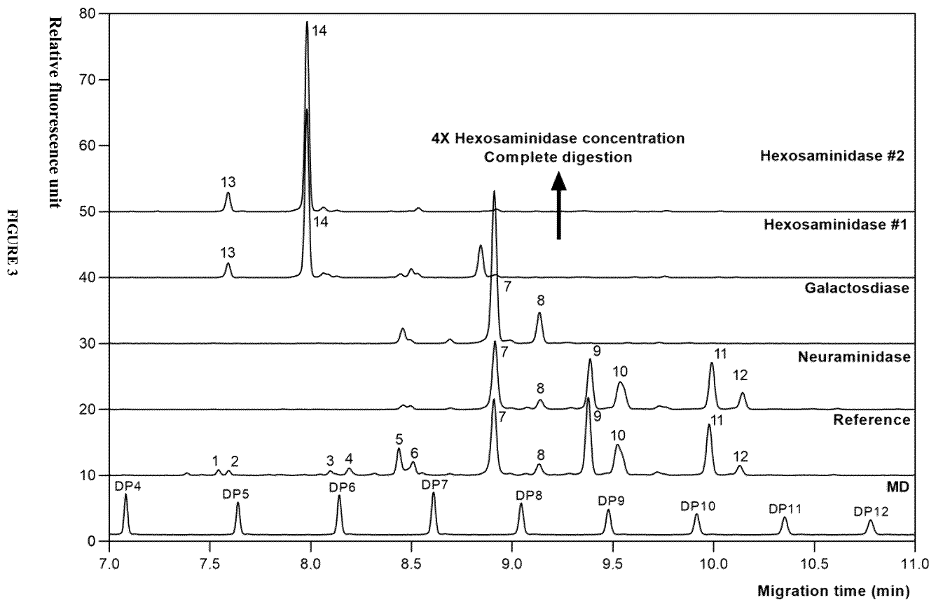

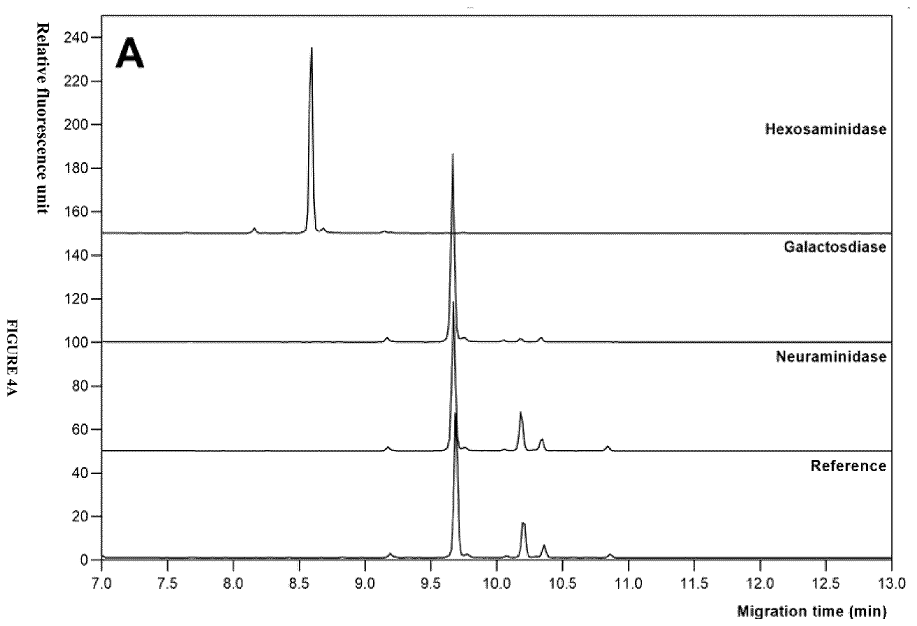

Although the individually used immobilized enzymes resulted complete digestion in 30 min using the temperature gradient method, the HEXase enzymes showed incomplete digestion when it was immobilized and was in mixture with the GALase and NANase enzymes. It is assumed that the three enzymes compete for the available Ni ions and the HEXase falls behind. By increasing the concentration of the HEXase to 0.6 μM (4×), complete reaction was obtained in 30 min using immobilized enzymes (FIG. 3).

Thus, the present inventors have found that when exoglycanases are applied in mixture for sequencing purposes adaptation of the concentration or ratio of the various types of exoglycanases may be adjusted to arrive at a complete digestion during the limited time-frame available for analysis.

This immobilization approach opens up the possibility for automation and for meeting special experimental needs where the immobilization of the enzymes is key.

### Test Method Using mAb and Biological Origin Samples

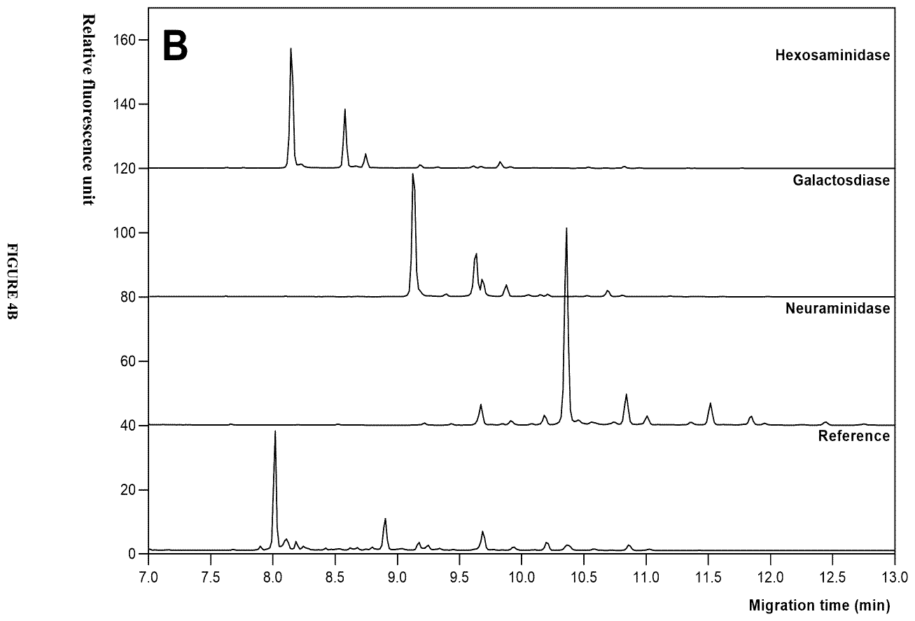

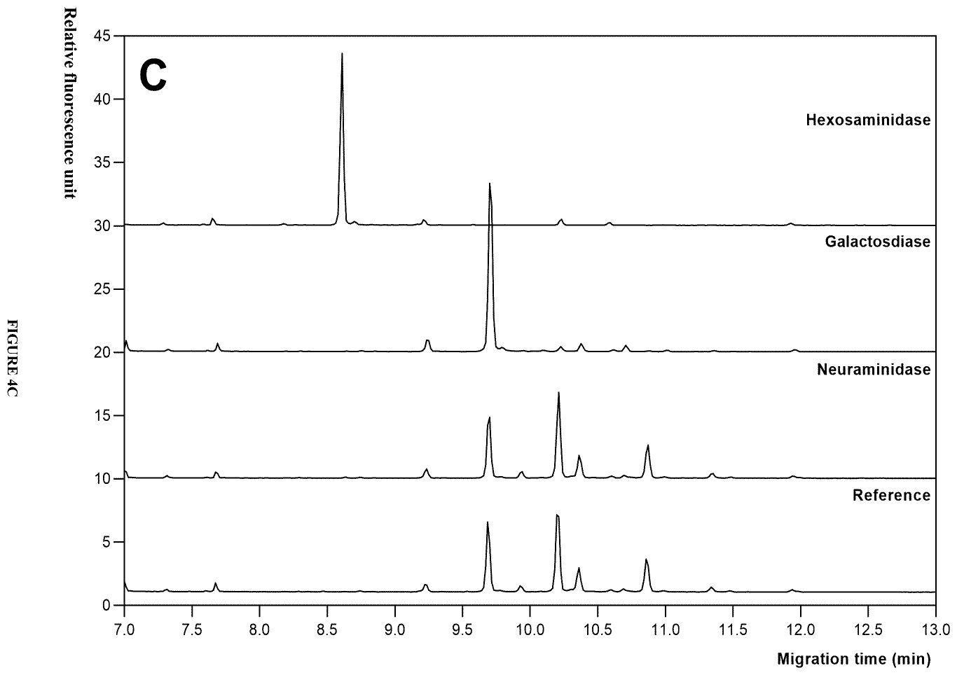

Other glycoprotein samples from different origin such as Daratumumab mAb glycans (panel A), human serum glycans (panel B) and Synagis mAb glycans (panel C) were tested with the premixed and co-dialyzed enzymes in aqueous phase and in immobilized form using the temperature gradient method for 30 min. Separation conditions were the same as in FIG. 2. In all cases, complete exoglycosidase digestion was obtained in the given timeframe (FIG. 4.).

### Glycan Sequencing by Separation Methods

As a separation method, for the analysis of the reaction mixtures capillary gel electrophoresis (CE) with laser-induced florescence (LIF) as detection was used for glycan sequencing.

CE-LIF is a well known technique and apply its variants is within the skills of a person skilled in the art. Such variants of the method are described e.g. in reviews [Lu, C. L. et al. Capillary Electrophoresis Separations of Glycans, Chem Rev 118(17) (2018) 7867-7885;]

The skilled person will understand that this method is highly preferred due to speed, reliability and small sample volume. Currently several analytical approaches are applied to identify the structure and linkage composition of glycans in biological samples, among others liquid chromatography (HPLC) or capillary electrophoresis (CE) separation w/o mass spectrometry (MS) analysis [Qin, W. et al. Alteration of Serum IgG Galactosylation as a Potential Biomarker for Diagnosis of Neuroblastoma, J Cancer 9(5) (2018) 906-913; Ashline, D. J. et al. Carbohydrate structural isomers analyzed by sequential mass spectrometry, Anal Chem 79(10) (2007) 3830-42; Reinhold, V. N. et al. Structural characterization of carbohydrate sequence, linkage, and branching in a quadrupole Ion trap mass spectrometer: neutral oligosaccharides and N-linked glycans, Anal Chem 70(14) (1998) 3053-9.] followed by glucose unit (GU) calculation [Mittermayr, S. and Guttman, A. Influence of molecular configuration and conformation on the electromigration of oligosaccharides in narrow bore capillaries, Electrophoresis 33(6) (2012) 1000-7; Jarvas, G. et al. Structural identification of N-linked carbohydrates using the GUcal application: A tutorial, J Proteomics 171 (2018) 107-115. and Jarvas, G. et al. Triple-Internal Standard Based Glycan Structural Assignment Method for Capillary Electrophoresis Analysis of Carbohydrates, Analytical Chemistry 88(23) (2016) 11364-11367.] are the matter of choice.

### Examples

### Materials and Methods

### Chemicals and Reagents

Water (HPLC grade), acetonitrile, dithiothreitol (DTT), ammonium acetate, acetic acid, sodium dodecyl sulfate (SDS), sodium cyanoborohydride (1 M in THF), ethylenediaminetetraacetic acid (EDTA), glycine, tetramethylethylenediamine (TEMED), ammonium persulfate (APS) and Nonidet P-40 (NP-40) were obtained from Sigma Aldrich (St. Louis, MO, USA). Aminopyrene-1,3,6-trisulfonic acid (APTS), the N-linked carbohydrate separation buffer (NCHO) and the M1 magnetic beads of the Fast Glycan Kit were from Sciex (Brea, CA, USA). The hIgG1 glycoprotein was from Molecular Innovations (Peary, MI, USA). The daratumumab (Darzalex) and palivizumab (Synagis) glycoproteins and the human serum were the courtesy of University of Debrecen (Debrecen, Hungary). PNGase F enzyme was produced in house but is also available from ThermoFisher Scientific or Gibco. Tris-HCL and NaCl were from VWR (Radnor, Pennsylvania, USA). NaH₂PO₄ was from Spektrum 3D (Debrecen, Hungary). Glycerol, Coomassie Brillant Blue R250 and imidazole were produced by Merck (Darmstadt, Germany). The 40% (37.5:1) acrylamide and bis-acrylamide solution was from Bio-Rad Laboratories (Hercules, California, USA) and boric acid from Scharlab (Debrecen, Hungary). Agarose was obtained from Nippon Genetics Europe (Duren, Germany).

### Exoglycosidase Enzyme Production

### Microorganisms, Vectors, Media, and Enzymes

Expression vectors pET23b and pET17b were purchased from Novagen (Madison, Wisconsin, US). Cloning E. coli strain TOP10 was from Invitrogen (Carlsbad, California, US). Restriction endonucleases AgeI HF, SacI HF, NdeI and XhoI, and the expression host SHuffle® T7 Express Competent E. coli was purchased from New England Biolabs (Ipswich, Massachusetts, USA). BL21-CodonPlus (DE3)-RIL E. coli cells originated from Agilent Technologies (Santa Clara, California, US). T4 DNA ligase was from Thermo Scientific (Waltham, Massachusetts, US). Cells were cultured in LB Broth medium and LB Agar (Scharlau, Barcelona, Spain). Ampicillin (Amp) and Chloramphenicol (Chl) were from Sigma Aldrich (St. Louis, Missouri, US). A 100 g/L Amp and a 30 g/L Chl stock solution was prepared and used in a 1,000-times dilution in the culture media. Coding DNA sequence of the functional part of neuraminidase (or sialidase) and hexosaminidase were synthesized by Biomatik (Cambridge, Ontario, Canada) and provided in pUC57 plasmid. Coding sequence of the functional part of β-galactosidase was synthesized by Twist Bioscience (San Francisco, California, US) and provided in pTwist plasmid. DNA sequencing was performed by Macrogen Europe (Amsterdam, the Netherlands).

### Construction of the Different Exoglycosidase Expressing Plasmids

Genes of neuraminidase (EC 3.2.1.18), hexosaminidase (EC 3.2.1.52) and β-galactosidase (EC 3.2.1.23) exoglycosidases were designed to enable the expressed proteins to immobilize by their HIS-tag. Considering the localization of the active center, the 6HIS tag, followed by a TEV protease cleavage site was fused to the C-terminal end of the enzymatically functional part of neuraminidase and hexosaminidase proteins.

The coding sequence of 39-990 polypeptide segment of neuraminidase (or sialidase, the native protein without its releasing signal peptide) from sia-AU gene from Paenarthrobacter ureafaciens (UniProt ID: Q5W7Q2) and the coding sequence of 34-1,280 polypeptide segment of β-N-acetylhexosaminidase (sequence of the native protein without its releasing signal peptide, the LPXTG recognition signal of sortase and the C-terminal peptidoglycan anchor) from strH gene from Streptococcus pneumoniae serotype 4 (UniProt ID: P49610) were codon optimized for E. coli and expanded with the SacI nuclease and the TEV protease cleavage site coding sequences at their 3′-end. The synthesized genes were provided in pUC57 between its EcoRV sites. These plasmids and the pET23b expression vector were digested by NdeI and XhoI restriction enzymes, purified from agarose gel and ligated by T4 ligase, fusing a C-terminal 6HIS-tag coding sequence to the designed gene. TOP10 E. coli cells were transformed by the ligation mixture and spread on LB Agar plates containing 100 μg/mL Ampicillin. Plasmid DNA from selected colonies was purified by Monarch Plasmid Miniprep Kit (New England Biolabs) and analyzed by restriction digestion and sequencing.

The coding sequence of 137-985 polypeptide segment of β-galactosidase (the catalytic domain of the native enzyme) from bgaA gene from Streptococcus pneumonia serotype 4 (UniProt ID: Q8DQP4) was first codon optimized for E. coli. The synthesized gene was provided in pTwist plasmid and digested by AgeI HF and SacI HF restriction enzymes. The pET17b plasmid was modified in our laboratory by the insertion of the coding sequences of an N-terminal 6-HIStidine tag, a TEV protease cleavage site, a folding enhancer glycine-serine-HIStidine (GSH) tripeptide and an AgeI restriction nuclease cleavage site by stepwise mutagenesis, after the start codon, resulting in the N-terminal polypeptide MHHHHHHENLYFQGSHTG fused to the catalytic domain of β-galactosidase after the AgeI cleavage site.

### Expression of the Exoglycosidase Proteins

The neuraminidase-TEV-6HIS (NANase) and hexosaminidase-TEV-6HIS (HEXase) coding pET23b plasmids were used to transform SHuffle T7 Competent E. coli cells applying the standard protocol recommended by the manufacturer. Protein expression was performed as follows: a 5.0 mL LB/Amp medium was inoculated and grown overnight at 30° C., 250 rpm. 0.5-0.75 L LB/Amp was supplemented with 1.0% (v/v) overnight culture and grown at 30° C. until OD₆₀₀ reached 0.6-0.8. Cell culture was induced by 0.4 mM IPTG and further incubated at 16° C., for 16 h. Cells were harvested by centrifugation at 10,000 g on a Heraus Biofuge primo R centrifuge instrument, for 30 min at 6.0° C., and subsequently frozen at −20° C. and then at −80° C. The 6HIS-TEV-GSH-galactosidase (GALase) coding pET17b plasmid was used to transform BL21-CodonPlus (DE3)-RIL E. coli competent bacteria. Cells were grown in 5.0 ml LB/Amp,Chl media overnight at 37° C., 250 rpm. 0.25 L LB/Amp, Chl was inoculated with 0.5% (v/v) overnight culture and grown at 37° C., 250 rpm. When OD₆₀₀ of the culture reached 0.6-0.8, it was induced with 0.5 mM IPTG and further incubated for 16 h at 30° C. The cells were harvested by centrifugation by 4,370 g on Heraus Multifuge 3S-R centrifuge instrument, for 30 min at 6.0° C., and frozen at −20° C. and then at −80° C.

### Purification of the Recombinant Exoglycosidases

Cell pellet from 0.5 L SHuffle/Neuraminidase-TEV-6HIS and 0.75 L SHuffle/HEXase producing cell culture was thawed and suspended in 10 mL and 15 mL ice-cold buffer ‘A’ (20 mM NaH₂PO₄, 500 mM NaCl, 25 mM imidazole, pH=7.5), respectively, supplemented with Complete protease inhibitor and incubated on ice for an hour. After addition of 2.0% (w/v) glass beads (diameter 0.5 mm, Sigma-Aldrich), cells were disrupted by sonication (Cole-Parmer, Vernon Hills, Illinois, USA) 10 times 30 sec or 45 sec on 10 W power. The remaining cell debris was settled by ultracentrifugation at 111,000 g in a Beckman Coulter Optima™ Max-XP Ultracentrifuge instrument using an MLA-80 fixed-angle rotor, for 30 min at 10° C. The supernatant was filtered through a 0.45 μm diameter syringe filter and applied directly to a pre-equilibrated 5 mL HiTrap Chelating Ni-affinity column (GE Healthcare, Chicago, Illinois, USA). NANase eluted at 50% B (buffer B: 20 mM NaH₂PO₄, 500 mM NaCl, 500 mM imidazole, pH=7.5) buffer, which corresponds to ca. 260 mM imidazole, while HEXase eluted in a single peak at 25% B (ca. 140 mM imidazole).

Cell pellet from 0.25 L cell culture producing BL21-CodonPlus (DE3)-RIL/GALase was thawed and suspended in 10 mL buffer ‘A’ supplemented with Complete protease inhibitor and incubated on ice for an hour. 2% (w/v) glass beads were added, and the cells disrupted by sonication 10 times 30 sec-45 sec on 10 W power. The remaining cell debris was settled by ultracentrifugation at 111,000 g on a Beckman Coulter Optima™ Max-XP Ultracentrifuge for 30 min at 10° C. The supernatant was filtered through a 0.45 μm diameter syringe filter and applied directly to a pre-equilibrated 5.0 mL HiTrap Chelating Ni-affinity column (GE Healthcare, Chicago, Illinois, USA). Main proportion of pure protein eluted at 125 mM buffered imidazole corresponding to 20% B buffer.

Purity of proteins in the eluted fractions were checked on 8-10% SDS PAGE, while protein concentration was calculated from the absorbance measured at 280 nm on a UV-Vis spectrophotometer (Jasco V-630, Tokyo, Japan) using the following parameters: the molecular weight MW=102.14 kDa and the molar extinction coefficient E=114,515 M⁻¹cm⁻¹ for NANase, MW=139.65 kDa and E=154,940 M⁻¹cm⁻¹ for HEXase and MW=98.37 kDa, E=175,560 M⁻¹cm⁻¹ for GALase.

### N-Glycan Sample Preparation

The N-glycan sample preparation was done using 10 μL of 10 mg/mL glycoprotein solution (hIgG1, Daratumumab, Synagis) or applying 10 μL human serum at 50-fold dilution based on Reider et al. [B. Reider, M. Szigeti, A. Guttman, Evaporative fluorophore labeling of carbohydrates via reductive amination, Talanta 185 (2018) 365-369.]. Briefly, the glycoprotein sample was denatured at 80° C. for 10 min using 2.0 μL of a denaturation mixture (12.5 mM DTT, 0.6% SDS and 0.06% NP40). After the incubation step, the sample was digested with 1.0 μL of PNGase F (0.1 mg/mL) in 20 μL of 20 mM AmAc at 37° C. for 2.0 h. After the digestion step, 20 μL of labeling solution was added (containing 6 mM of APTS, 100 mM of sodium cyanoborohydride in 1 M THF and 24% of acetic acid) and labeled overnight at 37° C. lid opened. Next, the excess dye was removed using 20 μL of tenfold concentrated M1 beads (Sciex) and 185 μL of acetonitrile alternately in total of 4 wash cycles. Then, the APTS labeled sample was eluted using 100 μL of HPLC grade water.

### Enzyme Pre-Mixture and Co-Dialyzation

Enzyme solutions were first prepared in mixtures containing enzymes at concentrations twenty times higher than those required for deglycosylation reactions (based on preliminary experiments targeting optimal enzyme concentration of complete enzymatic reaction at 37° C. in 1.0 hour). Three different compositions of solutions were prepared such as A) NANase only (0.3 mg/mL)—Mixture 1; B) NANase (0.3 mg/mL) and GALase (0.3 mg/mL)—Mixture 2 and C) NANase (0.3 mg/mL) and GALase (0.3 mg/mL) and HEXase (0.42 mg/ml)—Mixture 3. The exoglycosidase solutions were dialyzed together in a buffer of 20 mM Tris-HCl, 50 mM NaCl, pH=7.5 at 4.0° C. overnight. After dialysis, glycerol was added to the protein solutions at a final concentration of 50% in order to facilitate long term storage at −20° C. (up to 2 years).

### Enzyme Immobilization and Exoglycosidase Digestion

For all N-glycan sequencing using immobilized 6HIS tagged exoglycosidase enzymes, a 1 mL size Ni-IMAC pipette tips from PhyNexus (San Jose, CA, USA) were used with 40 μL bed volume. Pipetting was done automatically using more than 1000 pipette heads with the controller software (Capture-Purify-Enrich, Version 2.2.3, PhyNexus). The tips (three tips for each of the three enzyme mixtures) were washed prior to use with 1.0 mL of 20 mM ammonium acetate solution (pH=7.0) each using 1.4 mL/min aspiration and 2.8 mL/min dispensing speeds (700 μL total volume) for 2.0 min. Then, from each enzyme mixture 10 μL of solution was added to 90 μL of HPLC grade water (10× dilution) and immobilized on the pre-washed tips using 0.6 mL/min aspiration and 1.2 mL/min dispense speeds for 20 min (300 μL pipetting volume to ensure extensive contact of the enzyme and the resin). At last, the pipette tip was washed again with 1.0 mL of fresh ammonium acetate using the same conditions as the tip wash. In case if need for longer storage, the prepared tips were stored using 50% glycerol in HPLC grade water up to a month (tested time period) between 2-8° C. During the preparation of the tips, the 100 μL of sample from the N-glycan sample preparation was divided to 20 μL portions and was diluted to 100 μL with 10 mM ammonium acetate (pH=4.5). Then, the three prepared tips were used using the same conditions as for the immobilization for digestion applying a temperature gradient approach for 30 min. The aqueous phase reference digestion experiments were carried out using the same parameters, but in that case the same amount of enzyme was added in 5.0 μL volume to the sample.

### Glycan Sequencing by Capillary Gel Electrophoresis

All capillary electrophoresis separations were performed on PA 800 plus capillary electrophoresis system equipped with a laser-induced florescence (LIF) detection system with 488 nm excitation and 520 nm emission from Sciex. For all separation, NCHO gel was used as separation media and 50 μm ID bare fused silica capillary with 50 cm total length (40 cm effective length). The injection of the sample was done by applying 5.0 psi pressure for 5.0 second in case of every measurement. The temperature of capillary and the sample storage garage were both 20° C. The applied electric field strength during separation was 600 V/cm (30 kV) with reverse polarity (anode in the inlet end of capillary).

### INDUSTRIAL APPLICATION

Comprehensive analysis of the N-linked carbohydrates of glycoproteins is gaining high recent interest in both the biopharmaceutical and biomedical fields. Glycan sequencing has a highly increased importance nowadays and finds application in several fields of biological and medical research as well as diagnostics.

Carbohydrate sequencing though being a well-established method, still mostly practiced by cumbersome manual processes. The present invention is useful to streamline this process and adaptation thereof to an automated carbohydrate sequencing approach using the appropriate exoglycosidase enzymes in conjunction with the utilization of some of the features of advanced analysis method in particular a capillary electrophoresis (CE) instrument. Moreover immobilization of the exoglycosidase enzymes, in particular in subsets have the advantage in speeding up and simplifying the process wherein the product can be used more easily in the laboratory.

Carbohydrate sequencing finds application among others in research, diagnostics including personalized diagnostics [Hennig, R. et al. Towards personalized diagnostics via longitudinal study of the human plasma N-glycome, Biochim Biophys Acta 1860(8) (2016) 1728-38.] and in biological drug production for analysis and monitoring etc.

### REFERENCES

Archer-Hartmann S. A., L. M. Sargent, D. T. Lowry, L. A. Holland, Microscale exoglycosidase processing and lectin capture of glycans with phospholipid assisted capillary electrophoresis separations, Anal Chem 83(7) (2011) 2740-7.
Ashline D. J., A. J. Lapadula et al. Carbohydrate structural isomers analyzed by sequential mass spectrometry, Anal Chem 79(10) (2007) 3830-42.
Chen J., P. Shah, H. Zhang, Solid phase extraction of N-linked glycopeptides using hydrazide tip, Anal Chem 85(22) (2013) 10670-4.
Christensen S and Egebjerg J. Cloning, expression and characterization of a sialidase gene from Arthrobacter ureafaciens. Biotechnol Appl Biochem. 2005 June; 41(Pt 3):225-31. doi: 10.1042/BA20040144. PMID: 15461582.
Gabius H. J., The sugar code: Why glycans are so important, Biosystems 164 (2018) 102-111.
Gattu S., C. L. Crihfield, L. A. Holland, Microscale Measurements of Michaelis-Menten Constants of Neuraminidase with Nanogel Capillary Electrophoresis for the Determination of the Sialic Acid Linkage, Anal Chem 89(1) (2017) 929-936.
Guttman A et al.: Fast Glycan Sequencing Using a Fully Automated Carbohydrate Sequencer, Sciex, https://sciex.com/products/consumables/fast-glycan-analysis-and-labeling-for-the-pa-800-plus 2017-09-14
Guttman M., C. Váradi, K. K. Lee, A. Guttman, Comparative glycoprofiling of HIV gp120 immunogens by capillary electrophoresis and MALDI mass spectrometry, Electrophoresis 36(11-12) (2015) 1305-13.
Hennig R., S. Cajic, M. Borowiak et al. Towards personalized diagnostics via longitudinal study of the human plasma N-glycome, Biochim Biophys Acta 1860(8) (2016) 1728-38.
Iwamori M et al. Involvement of the C-terminal tail of Arthrobacter ureafaciens sialidase isoenzyme M in cleavage of the internal sialic acid of ganglioside GM1. J Biochem. 2005 September; 138(3):327-34. doi: 10.1093/jb/mvi126. PMID: 16169883
Jarvas G., M. Szigeti, A. Guttman, Structural identification of N-linked carbohydrates using the GUcal application: A tutorial, J Proteomics 171 (2018) 107-115.
Jarvas G., M. Szigeti, J. Chapman, A. Guttman, Triple-Internal Standard Based Glycan Structural Assignment Method for Capillary Electrophoresis Analysis of Carbohydrates, Analytical Chemistry 88(23) (2016) 11364-11367.
Kobata A. (2013). Exo- and endoglycosidases revisited. Proceedings of the Japan Academy. Series B, Physical and biological sciences, 89(3), 97-117. https://doi.org/10.2183/pjab.89.97
Krenková J., F. Foret, Immobilized microfluidic enzymatic reactors, Electrophoresis 25(21-22) (2004) 3550-63.
Lu G., C. L. Crihfield, S. Gattu, L. M. Veltri, L. A. Holland, Capillary Electrophoresis Separations of Glycans, Chem Rev 118(17) (2018) 7867-7885.
Mittermayr S., A. Guttman, Influence of molecular configuration and conformation on the electromigration of oligosaccharides in narrow bore capillaries, Electrophoresis 33(6) (2012) 1000-7.
Pluvinage B, et al. Conformational analysis of StrH, the surface-attached exo-β-D-N-acetylglucosaminidase from Streptococcus pneumoniae. J Mol Biol. 2013 Jan. 23; 425(2):334-49. doi: 10.1016/j.jmb.2012.11.005. Epub 2012 Nov. 12. PMID: 23154168.
Qin W., H. Pei, R. Qin, R. Zhao, J. Han, Z. Zhang, K. Dong, S. Ren, J. Gu, Alteration of Serum IgG Galactosylation as a Potential Biomarker for Diagnosis of Neuroblastoma, J Cancer 9(5) (2018) 906-913.
Reider B., M. Szigeti, A. Guttman, Evaporative fluorophore labeling of carbohydrates via reductive amination, Talanta 185 (2018) 365-369.
Sheeley D. M., V. N. Reinhold, Structural characterization of carbohydrate sequence, linkage, and branching in a quadrupole Ion trap mass spectrometer: neutral oligosaccharides and N-linked glycans, Anal Chem 70(14) (1998) 3053-9.
Sheldon R. A., S. van Pelt, Enzyme immobilisation in biocatalysis: why, what and how, Chem. Soc. Rev. 42(15) (2013) 6223-6235.
Singh A. K. et al. Unravelling the multiple functions of the architecturally intricate Streptococcus pneumoniae beta-galactosidase, BgaA PLoS Pathog. 10:e1004364-e1004364(2014)
Song T., S. Ozcan, A. Becker, C. B. Lebrilla, In-depth method for the characterization of glycosylation in manufactured recombinant monoclonal antibody drugs, Anal Chem 86(12) (2014) 5661-6.
Szigeti M., A. Guttman, Automated N-Glycosylation Sequencing Of Biopharmaceuticals By Capillary Electrophoresis, Sci Rep 7(1) (2017) 11663.
Szigeti M., A. Guttman, Automated N-Glycosylation Sequencing Of Biopharmaceuticals By Capillary Electrophoresis, Sci Rep 7(1) (2017) 11663. (ld. fent)
Temporini C., E. Perani, E. Calleri, L. Dolcini, D. Lubda, G. Caccialanza, G. Massolini, Pronase-Immobilized Enzyme Reactor: an Approach for Automation in Glycoprotein Analysis by LC/LC-ESI/MSn, Analytical Chemistry 79(1) (2007) 355-363.
Thomas N. Krogh, Torben Berg, and Peter Hojrup Protein Analysis Using Enzymes Immobilized to Paramagnetic Beads Analytical Biochemistry 274, (1999) 153-162
Váradi C., C. Jakes, J. Bones, Analysis of cetuximab N-Glycosylation using multiple fractionation methods and capillary electrophoresis mass spectrometry, J Pharm Biomed Anal 180 (2020) 113035.
Vliegenthart J. F., The complexity of glycoprotein-derived glycans, Proc Jpn Acad Ser B Phys Biol Sci 93(2) (2017) 64-86.
Wang F, Li R, Jian H et al. Design and Construction of an Effective Expression System with Aldehyde Tag for Site-Specific Enzyme Immobilization. Catalysts. 2020; 10(4):410
Yamagami M., Y. Matsui, T. Hayakawa, S. Yamamoto, M. Kinoshita, S. Suzuki, Plug-plug kinetic capillary electrophoresis for in-capillary exoglycosidase digestion as a profiling tool for the analysis of glycoprotein glycans, J Chromatogr A 1496 (2017) 157-162.
Yamamoto S., M. Ueda, M. Kasai, Y. Ueda, M. Kinoshita, S. Suzuki, A fast and convenient solid phase preparation method for releasing N-glycans from glycoproteins using trypsin- and peptide-N-glycosidase F (PNGase F)-impregnated polyacrylamide gels fabricated in a pipette tip, Journal of Pharmaceutical and Biomedical Analysis 179 (2020) 112995.

1. A set of exoglycosidase enzymes (exoglycosidases) for use in N-glycan sequencing, each one of the exoglycosidases having different exoglycosidase activities specific for cleaving different terminal carbohydrates from non-reducing end of a glycan, and each one of the exoglycosidases comprises a peptide tag for immobilization of said exoglycosidase on a matrix support, said peptide tag being present in a position of the exoglycosidase sequence wherein it does not affect exoglycosidase activity, wherein
said set of exoglycosidases comprises at least
a Neuraminidase, preferably alpha Neuraminidase, having the activity for cleaving a terminal sialic acid from a glycan and wherein said peptide tag is engineered to the C-terminal of the Neuraminidase,
a β-Galactosidase, wherein said peptide tag is engineered to the N-terminal of the β-Galactosidase and
a Hexosaminidase, wherein said peptide tag is engineered to the C-terminal of the Hexosaminidase, and
wherein the level of Hexosaminidase is increased relative to Neuraminidase and beta-Galactosidase so as to have hexosaminidase activity sufficient to achieve a complete cleavage of the terminal carbohydrate during a pre-determined time-period defined for each exoglycosidases in the set.

2. The set of exoglycosidases according to claim 1,
wherein the pre-determined time-period is at most 1.0 h, preferably at most 40 minutes. preferably at most half hour or 30 minutes, and wherein preferably the temperature range is 37° C. to 60° C.

3. The set of exoglycosidases according to claim 1,
wherein the pre-determined time-period is at most half hour or 30 minutes, and wherein the temperature range is 37° C. to 60° C.

4. The set of exoglycosidases according to claim 1, wherein said peptide tag is a metal-chelating peptide tag and the matrix support is a metal-comprising matrix support.

5. The set of exoglycosidases according to claim 1, wherein the peptide tag is a His-tag and the matrix support is a transition metal-comprising matrix support, said transition metal more preferably being selected from Cu²⁺, Ni²⁺, Zn²⁺, and Co²⁺.

6. The set of exoglycosidases according to claim 1, wherein each of said exoglycosidases are immobilized on said matrix support.

7. The set of exoglycosidases according to claim 1 comprising more than one subsets of exoglycosidases, each subsets being different in at least one exoglycosidase from each other subset(s), wherein a subset may comprise a single exoglycosidase,
within a subset each exoglycosidases having different exoglycosidase activities specific for cleaving different terminal carbohydrates from non-reducing end of a glycan,
wherein preferably in a subset each exoglycosidase is immobilized on the same matrix support.

8. The set of exoglycosidases according to claim 7 wherein each of said exoglycosidases are immobilized on said matrix support, and
wherein in a subset each exoglycosidase is immobilized on the same matrix support.

9. The set of exoglycosidases according to claim 1, wherein the exoglycosidases mixed together are dialyzed together (co-dialyzed) to reduce negative salt effect.

10. A method for glycan sequencing comprising,
carrying out simultaneously glycan cleavage reactions with multiple subsets of the exoglycosidases according to claim 1, wherein each of said exoglycosidases are immobilized on said matrix support and analyzing each of the reaction mixtures to obtain glycan sequence information.

11. The method according to claim 10, wherein the result of the glycan cleavage reactions is analyzed to obtain glycan sequence information by a separation method selected from the group consisting of HPLC, UPLC and capillary electrophoresis preferably with fluorescence detection, preferably by capillary electrophoresis with fluorescence detection.

12. The method according to claim 11, wherein the exoglycosidases mixed together are dialyzed together (co-dialyzed) to reduce negative salt effect in the capillary electrophoresis analysis.

13. A method for the preparation of a set of exoglycosidases according to claim 1, wherein
nucleic acid sequences encoding the exoglycosidases are provided, said exoglycosidases comprising at least a Neuraminidase, preferably alpha Neuraminidase, having the activity for cleaving a terminal sialic acid from a glycan, a β-Galactosidase and a Hexosaminidase,
nucleic acid sequences encoding the peptide tag are inserted into said nucleic acid sequences encoding the exoglycosidases, so that the peptide tag, once expressed, is present in a position of the exoglycosidase sequence wherein it does not affect exoglycosidase activity, wherein
said peptide tag is engineered to the C-terminal of the Neuraminidase,
said peptide tag is engineered to the N-terminal of the β-Galactosidase and
said peptide tag is engineered to the C-terminal of the Hexosaminidase,
each exoglycosidase is expressed in an expression system, preferably in a bacterial expression system,
each exoglycosidase is isolated,
preferably the isolation comprises a step wherein the exoglycosidase is bound to a chromatography matrix via the peptide tag,
the levels of the exoglycosidases are adjusted so that each exoglycosidase have exoglycosidase activity sufficient to achieve a complete cleavage of the terminal carbohydrate in a pre-determined temperature range and during a pre-determined time-period defined for each exoglycosidases in the set wherein the level of Hexosaminidase is increased relative to Neuraminidase and beta-Galactosidase so as to have hexosaminidase activity sufficient to achieve a complete cleavage of the terminal carbohydrate during a pre-determined time-period defined for each exoglycosidases in the set,
wherein preferably the pre-determined time-period is at most 1.0 h, preferably at most 40 minutes, preferably at most half hour or 30 minutes, and wherein preferably the temperature range is 37° C. to 60° C.
wherein at least a part of the exoglycosidases are mixed together to form a single cleavage reaction mixture.

14. The method for the preparation of a set of exoglycosidases according to claim 12 wherein a subset of exoglycosidases
for use in N-glycan sequencing, each one of the exoglycosidases having different exoglycosidase activities specific for cleaving different terminal carbohydrates from non-reducing end of a glycan, and each one of the exoglycosidases comprises a peptide tag for immobilization of said exoglycosidase on a matrix support, said peptide tag being present in a position of the exoglycosidase sequence wherein it does not affect exoglycosidase activity, wherein
said set of exoglycosidases comprises at least
a Neuraminidase, preferably alpha Neuraminidase, having the activity for cleaving a terminal sialic acid from a glycan and wherein said peptide tag is engineered to the C-terminal of the Neuraminidase,
a β-Galactosidase, wherein said peptide tag is engineered to the N-terminal of the β-Galactosidase and
a Hexosaminidase, wherein said peptide tag is engineered to the C-terminal of the Hexosaminidase, and
wherein the level of Hexosaminidase is increased relative to Neuraminidase and beta-Galactosidase so as to have hexosaminidase activity sufficient to achieve a complete cleavage of the terminal carbohydrate during a pre-determined time-period defined for each exoglycosidases in the set,
wherein the set of exoglycosidases comprises more than one subsets of exoglycosidases, each subsets being different in at least one exoglycosidase from each other subset(s), wherein a subset may comprise a single exoglycosidase,
within a subset each exoglycosidases having different exoglycosidase activities specific for cleaving different terminal carbohydrates from non-reducing end of a glycan,
wherein preferably in a subset each exoglycosidase is immobilized on the same matrix support,
are mixed together, and
the exoglycosidases mixed together are dialyzed together (co-dialyzed) to reduce negative salt effect on signal strength during capillary electrophoretic analysis.

15. The method for the preparation of a set of exoglycosidases according to claim 12 wherein the exoglycosidases are immobilized to a matrix support via the peptide tag,
wherein preferably
more than one subsets of exoglycosidases are mixed together and co-dialyzed,
each subsets being different in at least one exoglycosidase from each other subset(s), wherein a subset may comprise a single exoglycosidase,
within a subset each exoglycosidases having different exoglycosidase activities specific for cleaving different terminal carbohydrates from non-reducing end of a glycan.
and within a subset each exoglycosidase is immobilized on the same matrix support.

16. A kit for preparing an immobilized set of exoglycosidases as defined in claim 1, said kit comprising
a set of exoglycosidases as defined in claims 1 to 5 wherein said exoglycosidases are present in soluble form in buffer solution(s) as a stock exoglycosidase solution or set of stock exoglycosidase solutions,
a matrix support functionalized to be able for binding to or by the peptide tag engineered into the exoglycosidases,
optionally buffers and reagents to carry out immobilization, buffers for washing the matrix support before or after immobilization and storage buffers said storage buffers comprising preferably a stabilizing agent, preferably glycerin.
